(19) United States
(12) Patent Application Publication
(10) Pub. No.: US 2007/0071692 A1
(43) Pub. Date: Mar. 29, 2007

(54) Aerosol powder formulation

(76) Inventors: Roch Thibert, Mont-Royal (CA); Dale Meisner, Beaconsfield (CA); Joanna Rossi, St-Laurent (CA); Helen Tanfara, Pierrefonds (CA)

Correspondence Address: MERCK AND CO., INC, P O BOX 2000, RAHWAY, NJ, 07065-0907, US

(21) Appl. No.: 11/528,107

(22) Filed: Sep. 27, 2006

Related U.S. Application Data
(60) Provisional application No. 60/721,402, filed on Sep. 28, 2005.

(51) Int. Cl. A61K 9/14; A61K 31/4745

(52) U.S. Cl. 424/46; 514/300

(57) ABSTRACT

This invention relates to dry power aerosol formulations for use with a dry powder inhaler, the formulation comprising the PDE 4 inhibitor N-cyclopropyl-1-[3-(1-oxido-3-pyridinylethynyl)phenyl]-1,4-dihydro[1,8]naphthyridin-4-one-3-carboxamide:

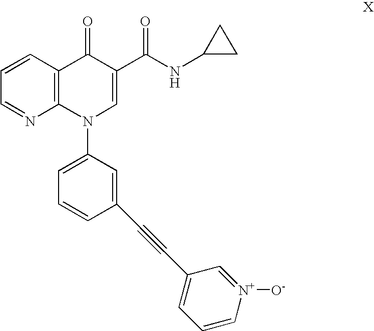

### FIELD OF THE INVENTION

The present invention relates to aerosol formulations for administration of a phosphodiesterase 4 (PDE 4) inhibitor. In particular, this invention relates to dry power aerosol formulations for use with a dry powder inhaler, the formulation comprising the PDE 4 inhibitor N-cyclopropyl-1-[3-(1-oxido-3-pyridinylethynyl)phenyl]-1,4-dihydro[1,8]naphthyridin-4-one-3-carboxamide: 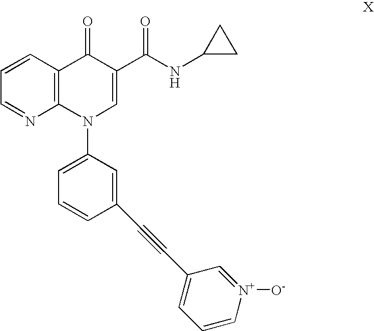
For purposes of this specification this compound will be called Compound X.

### BACKGROUND OF THE INVENTION

Drug delivery via the lung has the potential for higher therapeutic benefits to adverse side effects by avoiding gastrointestinal tract problems sometimes seen with oral delivery. In addition, the large surface area of the lung allows for good absorption of molecules into the bloodstream. See A. J. Hickey, Pharmaceutical Inhalation Aerosol Technology, Volume 54 (1992), 155-185.

Dry powder inhalers (DPI) are an alternate dosage form for inhalation approaches such as, nebulizers and metered dose inhalers (MDI). Powder inhaler (PI) systems can be viewed as consisting of three elements: the device, the formulation and an external force (e.g. supplied by the patient). The external force (e.g the patient) provides the energy for inhalation and the device creates turbulent forces which disperse the drug particles from weak agglomerates or from the surface of the carrier. A superior formulation may be one which utilizes a conventional, new or modified device, a conventional, new or modified excipient, and drug concentrations and processing steps that complement the selected/modified device. See D. Ganderton and N. M. Kassem, “Dry powder inhalers”, Advances in Pharmaceutical Sciences, Vol. 6 (1992) 165-191.

Conversion from a bulk powder to an aerosol is hindered by the cohesiveness of the particles. To facilitate dispersion and improve dose uniformity, an inert carrier, typically lactose, is added to the PI formulation. Ideally, the carrier should deposit in the upper airways of the patient while the micronized drug reaches the lung for absorption. See T. Srichana, G. P. Martin and C. Marriott, “On the relationship between drug and carrier deposition from dry powder inhalers in vitro”, International Journal of Pharmaceutics 167 (1998) 13-23. The surface of the carrier ought to possess adequate adhesion properties such that the blend does not segregate during handling and filling, but releases the drug during inhalation. See D. Ganderton and N. M. Kassem, supra.

### SUMMARY OF THE INVENTION

This invention relates to dry power aerosol formulations for use with a dry powder inhaler, the formulation comprising the PDE 4 inhibitor N-cyclopropyl-1-[3-(1-oxido-3-pyridinylethynyl)phenyl]-1,4-dihydro[1,8]naphthyridin-4-one-3-carboxamide:

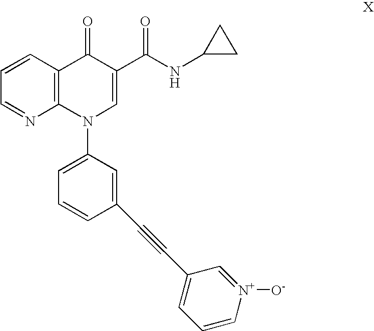

### BRIEF DESCRIPTION OF THE FIGURES

FIG. 1 Dose uniformity: Measured shot weight for Formulations A, B, and C (Target shot weight 25 mg.)

FIG. 2 Dose uniformity: Mass of Compound X recovered in the tested dosage unit sampling apparatus for formulations A, B, and C (Target Dose weight 1 mg)

FIG. 3 Aerodynamic size distribution for formulations A, B and C.

FIG. 4 Dose uniformity: Measured shot weight for formulations D, E and F (Target shot weight 25 mg).

FIG. 5 Dose uniformity: Measured shot weight for formulation G (Target shot weight 10 mg).

FIG. 6 Dose uniformity: Mass of Compound X recovered in the dosage unit sampling apparatus for formulations D, E, F and G (Target shot weight 1 mg).

### DETAILED DESCRIPTION OF THE INVENTION

In one aspect the invention is directed to a composition comprising Compound X 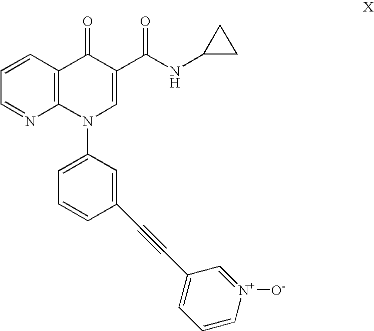
and
sieved lactose for inhalation
wherein Compound X is milled.

Within this aspect, there is a genus wherein the weight ratio of lactose to Compound X is from 20:1 to 30:1.

Within this aspect and genus there is a sub-genus wherein the weight ratio of lactose to Compound X is approximately 25:1.

Within this aspect, genus and sub-genus, there is a class wherein
Compound X is milled to a particle size of approximately 2-5 μm in diameter.

Within this aspect, genus, sub-genus and class, there is a sub-class wherein Compound X is jet milled.

Within this aspect, genus, sub-genus, class and sub-class there the weight of Compound X is from 0.2 mg to 2.5 mg.

Compound X, uses of the compound and methods of making same are disclosed in WO 03/018579, published Mar. 6, 2003 and WO2004/048377, published Jun. 10, 2004.

### EXAMPLES

In the studies described below the aerosol performance of micronized Compound X with three different grades of lactose made specifically for inhalation in an Spinhaler® is compared. The carriers studied were milled lactose for inhalation (Respitose™ ML003), sieved lactose for inhalation (Respitose SV003) and granulated lactose for inhalation (Respitose LS243). Each carrier was characterized for particle size, flow properties and morphology. Jet milled Compound X, with an average particle size of 2.8 microns was used in the studies. The reduction in particle size by milling the drug did not induce any change in crystal form or thermal properties when compared to the unmilled drug. Blends were manufactured with 4% w/w drug loading at a scale of 1 g. Capsules were then filled with 25 mg of blend to achieve a 1 mg nominal dose. Each blend was characterized for blend uniformity, dose uniformity and aerodynamic particle size distribution. Granulated lactose produced the weakest drug aerosolization compared to milled and sieved lactose. Drug dispersion was the best with milled lactose however the poor flow properties of the carrier resulted in variable shot weight. A 4% w/w formulation in sieved lactose was selected for in vivo animal studies since the fine particle respirable mass was similar to milled lactoose and better shot weight was achieved with sieved lactose. The selected formulation exhibited acceptable blend uniformity after the addition of a de-lumping step during preparation of the blend. The delivered shot weight was 92% of target with an in-vitro respirable fraction of 26% and an emitted dose of 34%. The formulation is considered appropriate for animal model studies use however optimization of the formulation and the blending characteristics will be pursued following results from the in-vivo animal studies.

Active Pharmaceutical Ingredient Description

Three jet milled samples of Compound X observed using X-ray powder diffraction (XRPD) and thermogravimetry (TGA) that the jet milled samples had similar properties to the unmilled lots. The samples retained their crystalline form. By SEM, it was observed that the jet milled drug was smaller in particle size compared to the unmilled drug, while maintaining the needle-like morphology. Drug particle size ranged from ca. 2-25 μm in length and ca. 2 μm in width with agglomerates up to 50 μm in diameter. Only one of the jet milled lots was used for the described studies below. A side by side comparison of the unmilled drug and the jet milled drug is shown in Table 1.

TABLE 1
Particle size of unmilled and jet milled Compound X

| | Unmilled API | Milled API | |
|---|---|---|---|
| | Optical microscopy | Microtrac data* | Aerosizer data |
| Mean (microns) | 11 | 2.794 | 2.832 |
| SD (microns) | 12 | 0.639 | 2.246 |
| 95% (microns) | 37 | 4.577 | 7.832 |

*After sonication for 60 seconds

Carriers Investigated

Three different grades of lactose were investigated as carriers for Compound A. The carriers studied were milled lactose for inhalation (Respitose™ ML003), sieved lactose for inhalation (Respitose SV003) and granulated lactose for inhalation (Respitose LS243). All carriers were supplied by DMV International.

Carrier Characterization

Each carrier was characterized for geometric diameter using an Aerosizer® LD and morphology using a JSM-5900LV scanning electron microscope. To assess carrier flow behavior, Carr's index was also obtained.

Formulation

All blends were prepared in the same manner by blending in a low shear tumbling blender (Turbula Type T2F ) for 15 minutes at 32 rpm. The blends contained 4% API and were manufactured at a scale of 1 g in a 4 ml glass amber bottle (50% fill volume). Then, 25 mg of blend, equivalent to 1 mg of drug, was weighed into each capsule (capsule size: 2LLC white opaque). The formulations are described in Table 2.

To assess blend uniformity, two capsules from each blend were opened, rinsed with solvent and assayed using a UV-Vis spectrophotometer. The solvent used for the DPI studies was a 60:40 mixture of methanol and water. The solvent was prepared in batches of 1000 ml. Six hundred milliliters of methanol was added to four hundred milliliters of water. The solution was then covered and allowed to cool to room temperature. To detect Compound X a calibration curve was developed using a UV-Vis spectrophotometer. In the 200 to 400 nm range, the maximum absorbance of Compound X was found to be 257 nm.

Capsules with 5 mg of drug only were also prepared to observe the behavior of Compound X in the Spinhaler without the aid of a carrier (Table 2).

TABLE 2
DPI formulations with 4% drug loading and different carriers

| | | Formulation | | | |
|---|---|---|---|---|---|
| | | A | B | C | Drug |
| | | (% | (% | (% | only |
| Ingredient | Function | w/w) | w/w) | w/w) | (% w/w) |
| Milled lactsoe | Carrier | 96 | — | — | — |
| Sieved lactose | | — | 96 | — | — |
| Granulated | | — | — | 96 | — |
| lactose | | | | | |
| Compound X | API | 4 | 4 | 4 | 100 |
| Batch size (g) | — | 1 | 1 | 1 | — |
| Shot weight | — | 25 | 25 | 25 | 5 |
| (mg) | | | | | |
| Dose (mg) | — | 1 | 1 | 1 | 5 |

Dose Uniformity Studies

Dose uniformity was determined using Apparatus B (Dosage Unit Sampling Apparatus—DUSA) at a flow rate of 100 L/min (test described in United States Pharmacopoeia (USP) 27 Chapter <601>). The USP recommends selecting a flow rate that creates a pressure drop of 4 kPa across the inhaler. With the Spinhaler, a 4 kPa pressure drop could not be achieved even at the maximum flow rate of 100 L/min. Based on the recommendations of Byron, et al., a flow rate of 100 L/min was selected since the Spinhaler is a low resistance device. See Michael Hindle and Peter R. Byron, “Dose emissions from marketed dry powder inhalers”, International Journal of Pharmaceutics 116 (1995)169-177. The test was run for 2.4 seconds in order to pull 4 L of air. After a shot had been delivered, all pieces of the DUSA including the mouthpiece adapter were rinsed with solvent. To determine the amount of drug retained in the inhaler, all pieces of the inhaler were rinsed with solvent including the interior of the capsule. The samples were then assayed using the UV-Vis spectrophotometer.

Shot weight was obtained by measuring the weight loss due to the actuation of the device. The device was tared, a “shot” was wasted in the DUSA and the device was re-weighed to obtain the delivered shot weight.

Dose and shot weight were deemed acceptable if they were within 75% to 125% of the theoretical values (USP <601>).

Aerodynamic Particle Size Distribution

The Anderson cascade impactor (Apparatus 3) was the device used to determine the aerodynamic size distribution. The impactor provided in vitro measurements of the fraction of the aerosol that has the potential to reach the alveolar region of the lung. This value is represented by the portion of particles below plate 2. The impactor was operated at 100 L/min for 2.4 seconds according to the method described in USP 27 <601>. Each impactor plate was coated with silicone grease (316 Dow Corning) to prevent particles from bouncing off the plates and returning to the air stream. Plates 6 and 7 were omitted since the test flow rate was greater than 60 L/min. All pieces of the impactor including the inhaler and capsule were rinsed with solvent and assayed using the UV-Vis spectrophotometer. The respirable portion was quantified by the in vitro respirable fraction (%RF) and fine particle respirable mass.

Dose uniformity and cascade impaction tests were carried out at controlled temperature (20-25° C.) and humidity (35% RH).

Investigation into a Blend De-Lumping Step

In an attempt to improve blend uniformity an investigation into a blend de-lumping step was carried out. Two different de-lumping methods were considered for this study: milling and geometric dilution.

Formulation

Blend de-lumping was investigated with sieved lactose at different batch sizes (1 g and 25 g) and drug loads (4% w/w and 10% w/w). The processing conditions are outlined in Table 3.

TABLE 3
Formulations to investigate a blend de-lumping step

| | | | | G |
|---|---|---|---|---|
| | D | E | F | (% w/w) |
| Ingredient | (% w/w) | (% w/w) | (% w/w) | ) |
| Sieved lactose | 96 | 96 | 96 | 90 |
| Compound X | 4 | 4 | 4 | 10 |
| Batch size (g) | 1 | 25 | 25 | 25 |
| Shot weight (mg) | 25 | 25 | 25 | 10 |
| Dose (mg) | 1 | 1 | 1 | 1 |
| De-lumping method | Milling | Geometric | Milling | Milling |
| | | dilution | | |
| Final mixing time (min) | 2 | 6 | 1 | 1 |

Blends D (4% API), F (4% API) andG (10% API) were de-lumped using a milling step at a scale of 1 g, 25 g and 25 g, respectively. First, sieved lactose and Compound X were added to a 4 ml or 4 oz glass amber bottle (depending on the batch size) in order to achieve approximately 50% fill volume. The blends were then mixed in a low shear tumbling blender mixer for 15 minutes at 32 rpm. The blends were passed through a comill using a 0.016″ flat screen and square impeller at 29 rpm. The de-lumped blend was then blended in the mixer at 32 rpm for a duration of 1 to 2 minutes. For the 4% formulations, 25 mg of blend was weighed into each capsule in order to achieve 1 mg of drug per capsule. For the 10% formulation, 10 mg of blend was weighed into each capsule.

Formulation E (4% API) was prepared using a geometric dilution step at a scale of 25 g. The drug was sandwiched between two layers of lactose and carefully triturated in a mortar and pestle using low shear force. The contents of the mortar was emptied into a 4 oz. glass amber bottle and mixed in a mixer for 6 minutes at 32 rpm. Then, 25 mg of blend, equivalent to 1 mg of drug, was weighed into each capsule.

To assess blend uniformity, two capsules from each blend were opened, rinsed with solvent and assayed using a UV-Vis spectrophotometer. The aerodynamic particle size was also determined.

Results and Discussion

Carrier Selection

Carrier Characterization

The flow properties and mean particle sizes of variuos lactose grades are summarized in Table 4. Similar flow properties were observed between sieved lactose and granulated lactose. Considerably poorer flow behavior was seen with milled lactose. Mean particle size was comparable between milled and sieved lactose however granulated lactose was slightly larger.

TABLE 4
Mean particle size and flow properties of various carriers

| | Geometric diameter (μm) | | Carr's index (%) |
|---|---|---|---|
| Excipient | Mean size | Std. dev. | Mean |
| Milled lactose | 35 | 1.5 | 52 |
| Sieved lacttose | 41 | 1.4 | 31 |
| Granulated lactose | 59 | 1.6 | 35 |

Scanning Electron Microscopy micrographs for the three carriers and the micronized drug were obtained. From the micrographs, it was observed that granulated lactose had more surface porosity than milled or sieved lactose. Needle-like particles were observed for the micronized drug, which were similar to the unmilled GMP lots.

Formulation

Blend uniformity results for formulations A, B and C are summarized in Table 5. It was observed that the amount of drug recovered was low for all blends. In addition, drug recovery in capsules A and B was considerably higher then C. The variable and low recovery may be due to poor blend uniformity and/or segregation during sampling and handling.

TABLE 5
Individual capsule assay for formulations A, B and C

| | | | | Mass of Compound X |
|---|---|---|---|---|
| | Drug load | Batch size | Capsule | recovered in capsule |
| Formulation | (% w/w) | (g) | number | (mg) |
| A | 4 | 1 | 1 | 0.79 |
| | | | 2 | 0.90 |
| B | 4 | 1 | 1 | 0.87 |
| | | | 2 | 0.77 |
| C | 4 | 1 | 1 | 0.26 |
| | | | 2 | 0.28 |

Dose Uniformity Studies

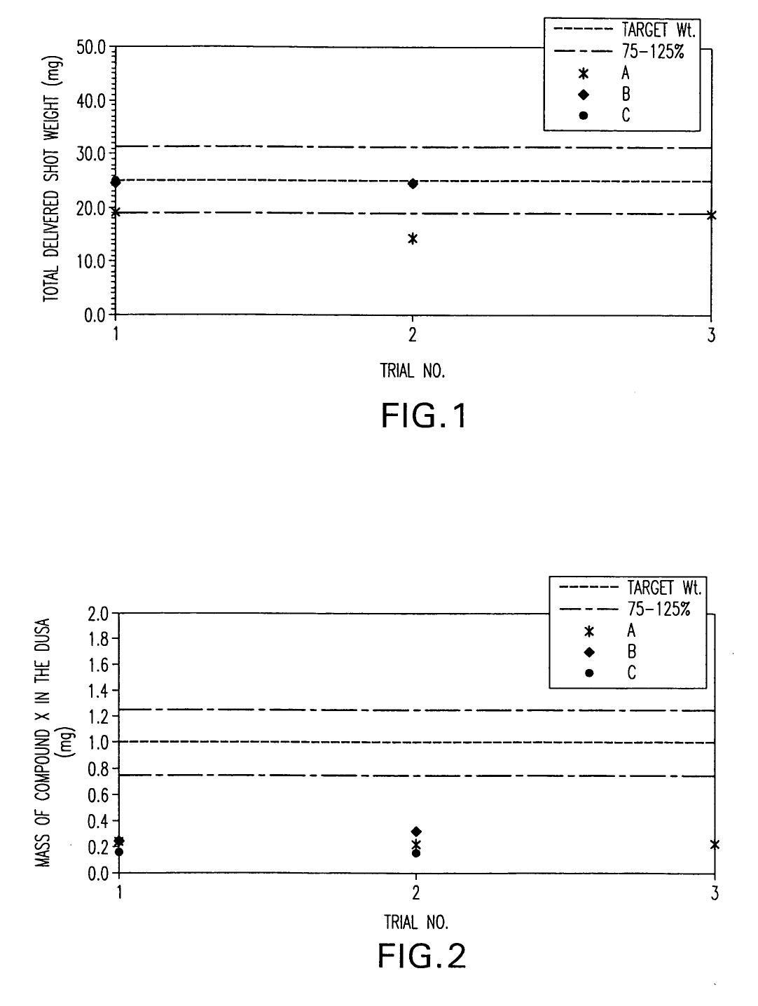

Dose uniformity results for formulations A, B and C are summarized in Table 6. It was observed that formulations B and C were on target for shot weight however formulation A was at or below the lower limit for acceptable shot weight, which may be attributed to the poor flow properties of the milled lactose FIG. 1. The average shot weights measured for B and C were 24.6±0.1 mg and 24.6±0.5 mg, respectively compared to 17.4±2.8 mg for A.

TABLE 6
Dose uniformity results for formulations A, B and C

| | | | | | Amount of Compound | | |
|---|---|---|---|---|---|---|---|
| | | Drug | | Shot | X recovered (mg) | | |
| Formu- | | load | Trial | weight | | In- | |
| lation | Carrier | (% w/w) | no. | (mg) | DUSA | haler | Total |
| A | Milled | 4 | 1 | 19.1 | 0.24 | 0.50 | 0.74 |
| | lactose | | 2 | 14.2 | 0.22 | 0.98 | 1.19 |
| | | | 3 | 18.9 | 0.23 | 0.67 | 0.90 |
| B | Sieved | 4 | 1 | 24.5 | 0.24 | 0.38 | 0.62 |
| | lactose | | 2 | 24.6 | 0.32 | 0.66 | 0.98 |
| C | Granu- | 4 | 1 | 24.9 | 0.16 | 0.06 | 0.22 |
| | lated | | 2 | 24.2 | 0.15 | 0.09 | 0.24 |
| | Lactose | | | | | | |
| Drug | — | 100 | 1 | 0.7 | 1.01 | 3.27 | 4.28 |
| only | | | 2 | 0.6 | 0.91 | 3.36 | 4.27 |

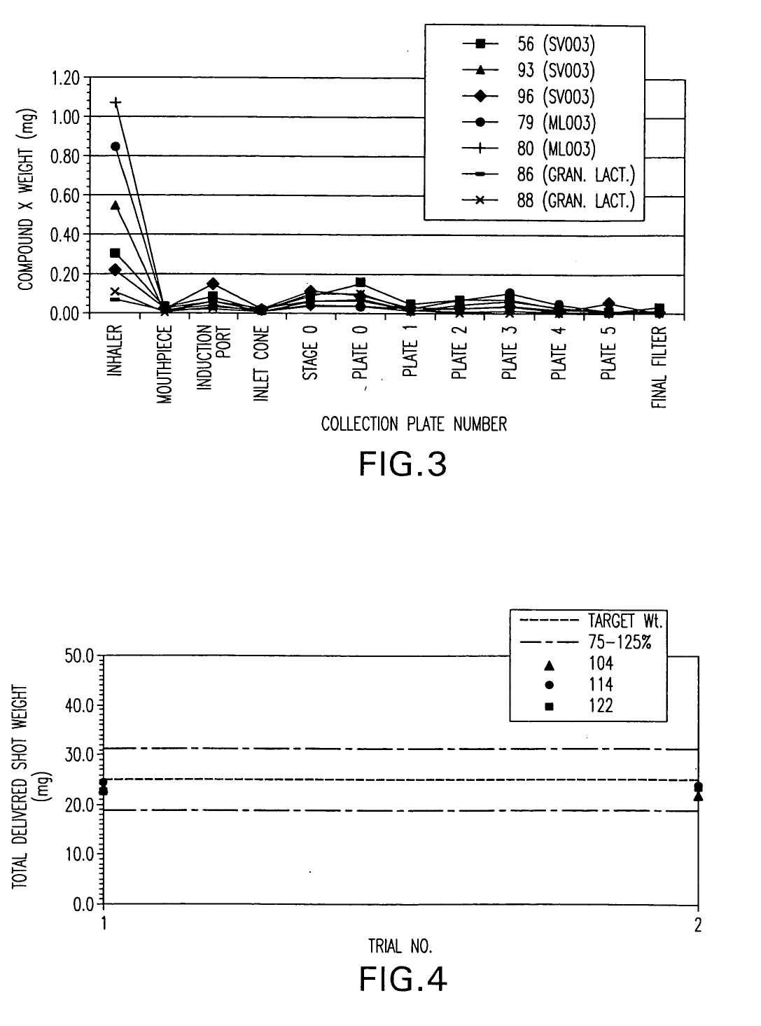

For all formulations, dose weight was well below the target value of 1 mg FIG. 3. The average amount of drug measured in the DUSA for formulations A, B and C was 23%, 28% and 16% of the nominal dose, respectively. For formulation C, the low mass of drug recovered in the DUSA was probably due to the 23% total drug recovery as a result of blend uniformity issues. To remove the effect of blend uniformity, the emitted dose of formulations A, B and C will be compared in terms of the amount of drug measured in the DUSA divided by the total amount of drug recovered in the system (DUSA+inhaler). Therefore, the average amount of drug measured in the DUSA for formulations A, B and C was 25%, 36% and 68% of the total recovered dose, respectively. With only drug and no carrier, approximately 23% of the 5 mg nominal dose was recovered in the DUSA, which demonstrates the poor flowability of the drug in the Spinhaler. Only formulations B and C improved the flow of drug particles out of the inhaler as seen by the increased emitted doses. The emitted dose was considerably higher in formulation C. One possible explanation is that granulated lactose (formulation C) possessed a much more porous surface than milled lactose (formulation A) and sieved lactose (formulation B) resulting in stronger interparticulate bonds due to the entrapment of the fine drug particles within the surface cracks and dimples. The stronger interparticle interactions formed with granulated lactose allowed more drug to be drawn out of the capsule with the carrier leaving less drug behind in the inhaler. The surfaces of milled lactose (formulation A) and sieved lactose (formulation B) were smoother making it more difficult for the drug to interact with lactose (see SEM micrographs). In addition to the surface properties of milled lactose, the poor flow properties of the carrier may have contributed to the low emitted dose observed in formulation A.
Aerodynamic Particle Size Distribution

The aerodynamic particle size distribution data for formulations A, B and C are shown in Table 7. The mean respirable fraction was 54%, 30% and 9% for formulations A, B and C, respectively. In addition, the mean fine particle mass was 0.18±0.06 mg, 0.14±0.04 mg and 0.02±0.01 mg for A, B and C, respectively. The results demonstrate that the drug disperses to the greatest extent in formulation A and the least in formulation C. As mentioned previously, the results can be explained by the greater interparticle interactions formed in formulation C due to the higher surface porosity.

With only 5 mg of drug and no carrier the greatest respirable portion was achieved with a respirable fraction of 65% and a mean fine particle mass of 0.62±0.04 mg.

TABLE 7
Cascade impaction results for formulations A, B and C

| | | | Target | | Fine | |
|---|---|---|---|---|---|---|
| | | Drug | dose | In vitro | particle | Emitted |
| Formu- | Batch | load | weight | respirable | mass | Dose |
| lation | size (g) | (% w/w) | (mg) | fraction (%) | (mg) | (mg) |
| A | 1 | 4 | 1 | 57 | 0.22 | 0.38 |
| | | | | 51 | 0.14 | 0.26 |
| B | 1 | 4 | 1 | 33 | 0.18 | 0.56 |
| | | | | 31 | 0.10 | 0.31 |
| | | | | 25 | 0.13 | 0.52 |
| C | 1 | 4 | 1 | 13 | 0.02 | 0.19 |
| | | | | 5 | 0.01 | 0.25 |
| Drug | — | 100 | 1 | 62 | 0.59 | 0.95 |
| only | | | | 68 | 0.64 | 0.95 |

Investigation into a Blend De-Lumping Step

To improve blend uniformity a blend de-lumping step was investigated. Two de-lumping methods were considered. The first was milling through a comill and the second was geometric dilution in a mortar and pestle. The results of these approachs are summarized in the following sections.

Formulation

Blend uniformity results are summarized in Table 8. It was observed that all blends were uniform however drug recovery was low for formulation 104 which may be due to scaling. One gram of blend was too small for the comil, which resulted in high material loss (24% of the blend was lost due to milling). Increasing the batch size improved drug recovery (see blend 122). At a 25-g scale, both milling and geometric dilution improved blend uniformity.

TABLE 8
Individual capsule assay for formulations
37, 104, 114, 122 and 131

| | | | | | Mass of |
|---|---|---|---|---|---|
| | Drug | Batch | Additional | | Compound X |
| | load | size | processing | Capsule | in capsule |
| Blend ID | (% w/w) | (g) | steps | number | (mg) |
| 37 | 4 | 1 | — | 1 | 0.87 |
| (formu- | | | | 2 | 0.77 |
| lation B) | | | | | |
| 104 | 4 | 1 | Milling | 1 | 0.49 |
| | | | | 2 | 0.53 |
| 114 | 4 | 25 | Geometric | 1 | 1.09 |
| | | | dilution | 2 | 1.10 |
| 122 | 4 | 25 | Milling | 1 | 1.08 |
| | | | | 2 | 1.08 |
| 131 | 10 | 25 | Milling | 1 | 1.09 |
| | | | | 2 | 1.08 |

Dose Uniformity Studies

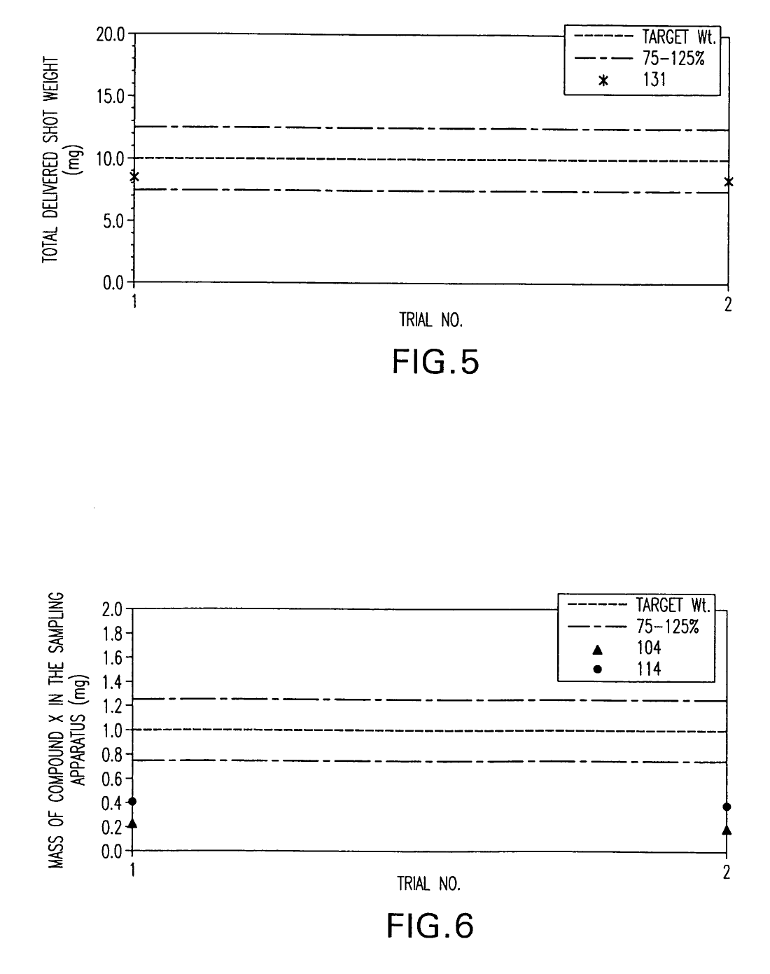

Dose uniformity results are summarized in Table 9. It was observed that all formulations were within 75 to 125% of the target shot weight FIG. 4 and FIG. 5. Average shot weights for the 4% w/w blends 104, 114 and 122 were 22.9±1.1 mg, 24.0±0.4 mg and 23.1±0.7 mg, respectively. Shot weight was slightly lower for the 10% formulation at 85% of the target value. This result may be due to the poorer flow properties of the higher drug load formulation. Other studies on Compound X demonstrated that flow properties decreased as drug load increased.

For all formulations, dose weight was outside the acceptable limit of 75% to 125% of the nominal dose FIG. 6. Dose recovery in the DUSA was similar to formulation B (37) for all blends. The emitted dose was slightly higher for blend 114. One possible explanation is that stronger interparticle interactions were formed between the drug and carrier during trituration. The stronger adhesion would allow more drug to leave the inhaler with the carrier.

TABLE 9
Dose uniformity results for formulations
104, 114, 122 and 2105-131

| | Drug | | | | Amount of Compound | | |
|---|---|---|---|---|---|---|---|
| | load | Batch | Additional | Shot | X recovered (mg) | | |
| | (% | size | processing | weight | | In- | |
| Blend ID | w/w) | (g) | steps | (mg) | DUSA | haler | Total |
| 37 | 4 | 1 | — | 24.5 | 0.24 | 0.38 | 0.62 |
| (formu- | | | | 24.6 | 0.32 | 0.66 | 0.98 |
| lation B) | | | | | | | |
| 104 | 4 | 1 | Milling | 23.6 | 0.24 | 0.19 | 0.43 |
| | | | | 22.1 | 0.19 | 0.27 | 0.46 |
| 114 | 4 | 25 | Geometric | 24.2 | 0.40 | 0.41 | 0.82 |
| | | | dilution | 23.7 | 0.38 | 0.41 | 0.79 |
| 122 | 4 | 25 | Milling | 22.6 | 0.31 | 0.56 | 0.87 |
| | | | | 23.6 | 0.37 | 0.50 | 0.87 |
| 131 | 10 | 25 | Milling | 8.5 | 0.21 | 0.57 | 0.78 |
| | | | | 8.4 | 0.25 | 0.60 | 0.85 |

Aerodynamic Particle Size Distribution

Aerodynamic particle size data generated by the Anderson cascade impactor is presented in Table 10. It was observed that introducing a blend de-lumping step, both milling and geometric dilution, decreased the respirable portion. This result may be explained by the greater drug/carrier interparticle interactions created as a result of milling and/or geometric dilution. Drug dispersion was lower with geometric dilution compared to milling. As mentioned previously, this result may be explained by the greater shear force exerted on the particles during trituration, which caused the drug to adhere more to the carrier particles.

TABLE 10
Cascade impaction results for formulations 104, 114, 122 and 131

| | | | | Drug | In vitro | Fine |
|---|---|---|---|---|---|---|
| | | Additional | Batch | load | respirable | particle |
| | | processing | size | (% | fraction | mass |
| Blend ID | Carrier | steps | (g) | w/w) | (%) | (mg) |
| 37 | SDieved | — | 1 | 4 | 33 | 0.18 |
| (formu- | lactose | | | | 31 | 0.10 |
| lation B) | | | | | 25 | 0.13 |
| 104 | Sieved | Milling | 1 | 4 | 28 | 0.09 |
| | lactose | | | | 27 | 0.08 |
| 114 | Sieved | Geometric | 25 | 4 | 14 | 0.06 |
| | lactose | dilution | | | 15 | 0.06 |
| 122 | Sieved | Milling | 25 | 4 | 22 | 0.09 |
| | lactsoe | | | | 30 | 0.09 |
| 131 | Sieved | Milling | 25 | 10 | 21 | 0.08 |
| | lactose | | | | 19 | 0.09 |
| | | | | | 23 | 0.08 |

Conclusion

An investigation into the aerosol performance of Compound X with different grades of lactose at 4% w/w drug loading demonstrated that sieved lactose was the most suitable carrier of the three choices. Granulated lactose produced the weakest drug aerosolization compared to milled and sieved lactsoe. Drug dispersion was the best with milled lactose however the poor flow properties of the carrier resulted in variable shot weight. Sieved lactsoewas chosen since the fine particle respirable mass was similar to milled lactose and better shot weight was achieved with sieved lactose. Blend uniformity issues were encountered with all carriers. The introduction of a blend de-lumping step improved blend uniformity however decreased the respirable portion.

A 4% w/w drug load formulation in sieved lactose with a milling step during blend preparation was found to possess a combination of superior properties. The delivered shot weight was 92% of target with an in-vitro respirable fraction of 26% and an emitted dose of 34%.

What is claimed is:

1. A composition comprising Compound X 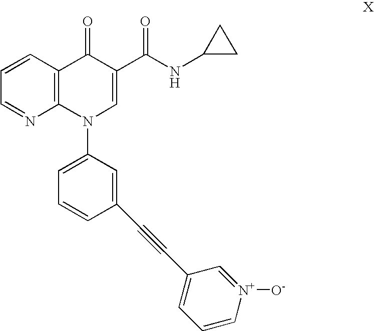
and
sieved lactose for inhalation
wherein Compound X is milled.

2. A composition accord to claim 1 wherein the weight ratio of lactose to Compound X is from 10:1 to 100:1.

3. A composition according to claim 2 wherein the weight ratio of lactose to Compound X is approximately 25:1.

4. A composition according to claim 1, claim 2 or claim 3 wherein Compound X is milled to a particle size of approximately 2-5 μm in diameter

5. A composition according to claim 1, claim 2, claim 3 or claim 4 wherein Compound X is jet milled.

6. A composition according to claim 1, claim 2, claim 3, claim 4 or 5 wherein the weight of Compound X is from 0.2 mg to 2.5 mg.
